(19) United States
(12) Patent Application Publication
(10) Pub. No.: US 2010/0016967 A1
(43) Pub. Date: Jan. 21, 2010

(54) RESORBABLE MEDICAL IMPLANTS AND RELATED METHODS

(76) Inventors: Arnold-Peter C. Weiss, Barrington, RI (US); Jignesh B. Patel, Newtown, PA (US); Karishma Vohra, Cherry Hill, NJ (US); Namrata Desai, Bridgewater, NJ (US); Ronald T. Ingram, Encinitas, CA (US); Mark Spilker, Cranbury, NJ (US); Timothy J. Pryor, Yardley, PA (US); Heather Beam-Brennan, Red Bank, NJ (US)

Correspondence Address: JENKINS, WILSON, TAYLOR & HUNT, P. A., Suite 1200 UNIVERSITY TOWER, 3100 TOWER BLVD.,, DURHAM, NC, 27707, US

(21) Appl. No.: 12/501,089

(22) Filed: Jul. 10, 2009

Related U.S. Application Data
(60) Provisional application No. 61/134,702, filed on Jul. 11, 2008.
(60) Provisional application No. 61/219,508, filed on Jun. 23, 2009.

(51) Int. Cl. A61F 2/28; B29C 69/00; B32B 37/02

(52) U.S. Cl. 623/16.11; 264/241; 156/192

(57) ABSTRACT

Biocompatible, resorbable medical implants and related methods are disclosed in which the implant can be made of a collagen matrix for filling a space after partial or total resection of the trapezium bone of the wrist. The resorbable medical implant can include a plurality of surface projections, and the collagen matrix can be folded or spirally wound about itself to form a spiral roll.

### RELATED APPLICATIONS

The presently disclosed subject matter claims priority to U.S. Provisional Patent Application Ser. No. 61/134,702, filed Jul. 11, 2008, and to U.S. Provisional Patent Application Ser. No. 61/219,508, filed Jun. 23, 2009, the disclosures of which are incorporated herein by reference in their entireties.

### TECHNICAL FIELD

The subject matter disclosed herein relates generally to resorbable medical implants and related methods. More particularly, the subject matter disclosed herein relates to joint spacer implants made of resorbable materials and related methods.

### BACKGROUND

The carpometacarpal (CMC) joint is among the main targets for osteoarthritis of the hand. Many operative procedures have been advocated for the treatment of this disease, with an objective of obtaining a painless, stable, and mobile joint.

The current state of care includes silicone interposition, implants made of titanium or a metallic/ceramic combination, and autogenous graft CMC arthroplasty procedures. These options have disadvantages that make them undesirable for this purpose. Silicone implants exhibit high complication rates related to silicone particulation. Titanium and metallic/ceramic implants are generally perceived to be too hard and can damage adjacent bone structures.

With regard to autogenous CMC arthroplasty, there are two fundamental types of procedures practiced today: CMC arthroplasty with tendon interposition (TI) and CMC arthroplasty with ligament reconstruction and tendon interposition (LRTI). Both procedures involve excising the trapezium bone, and then maintaining the CMC joint space through various methods of ligament reconstruction and/or tendon interposition. It is thought that both the ligament reconstruction and tendon interposition act to preserve the thumb height while providing a scaffold for soft tissue ingrowth.

The TI procedure begins with exposing and resecting the entire trapezium and any osteophytes. Then, through a second incision, the flexor carpi radialis tendon is incised longitudinally in its midportion. With the distal attachment preserved, the tendon strip is rolled into a ball, sutured to itself, and inserted into the defect of the trapezium excision. The tendon ball is sutured into place, and the joint capsule/soft tissues are closed over it. A Kirschner wire (k-wire) is temporarily implanted to stabilize the joint.

LRTI surgery involves partial or complete resection of the trapezium and recreation of the palmar oblique ligament using the distally based radial half of the flexor carpi radialis to reconstruct ligamentous support for the metacarpal. A supporting ligamentous sling is made by passing the tendon through the center of the base of the metacarpal and its ulnar cortex, in order to maintain its length and prevent radial subluxation. The remaining tendon is folded and placed into the area of the absent trapezium, similar to the TI procedure. As necessary, k-wires are used to temporarily stabilize the joint. Finally, the soft tissues are closed. There are numerous variations of the LRTI procedure that involve different methods of ligament reconstruction and the degree of tendon interposition. A surgeon's decision to employ such variations is dependent upon his or her personal preferences, training, and patient factors.

In comparing the clinical results of TI and LRTI procedures, both have similar outcomes. To date, no study has shown a long-term advantage of one procedure over the other. With such equivalent outcomes, the individual surgeon's procedure of choice is largely determined by their clinical training and subsequent familiarity with the surgical technique.

Although autogenous CMC arthroplasty surgery (both TI and LRTI techniques) has been shown to be a safe and effective treatment, there continues to be a large volume of research for alternatives, in part because extended procedure time and associated graft harvest-site morbidity associated with these procedures can be problematic. The focus of the research tends to be on variations of ligament reconstruction and the exploration of other materials to fill the void left by the excised trapezium.

There remains a need for a space-filling device which can help save operating time and reduce the morbidity associated with tendon harvesting.

### SUMMARY

The presently disclosed subject matter provides resorbable medical implants, and methods relating thereto, for replacing a bone of small size and for filling voids between bones. For example, an implant can be used as a CMC joint spacer to treat thumb osteoarthritis in the joint between the first metacarpal and the trapezium (a carpal bone). The implant can comprise a resorbable material, for example, collagen. Specifically, in some embodiments, the implant can comprise bovine type I collagen and can serve as an interpositional spacer in the thumb basal joint.

The presently disclosed subject matter can further provide a resorbable space-filling implant to address arthroplasty procedures for the osteoarthritic CMC joint of the thumb. In this regard, the implant can be used to save operating time and reduce the morbidity associated with tendon harvesting, while providing decreased pain and increased mobility and function.

### BRIEF DESCRIPTION OF THE DRAWINGS

The features and advantages of the present subject matter will be more readily understood from the following detailed description which should be read in conjunction with the accompanying drawings that are given merely by way of explanatory and non-limiting example, and in which:

FIGS. 1A and 1B are perspective views of a resorbable medical implant according to embodiments of the presently disclosed subject matter;

FIGS. 2A through 2I illustrate steps in the formation of a resorbable medical implant according to a rolled embodiment of the presently disclosed subject matter;

FIG. 3 is an illustration of a scanning electron micrograph at 35× magnification of a collagen matrix for use in the process of making a resorbable medical implant according to one aspect of the presently disclosed subject matter;

FIG. 4 shows additional illustrations of scanning electron micrographs of the collagen matrix;

FIG. 5 is a posterior view of the palm bones of a hand;

FIGS. 6A through 6C are posterior views of the palm bones of a hand with a resorbable medical implant oriented in different directions according to embodiments of the presently disclosed subject matter;

FIGS. 7A and 7B illustrate steps in the formation of a resorbable medical implant according to a first folded embodiment of the presently disclosed subject matter;

FIGS. 8A through 8C illustrate steps in the formation of a resorbable medical implant according to a second folded embodiment of the presently disclosed subject matter; and

FIG. 9 illustrates a resorbable medical implant according to a stacked embodiment of the presently disclosed subject matter.

### DETAILED DESCRIPTION

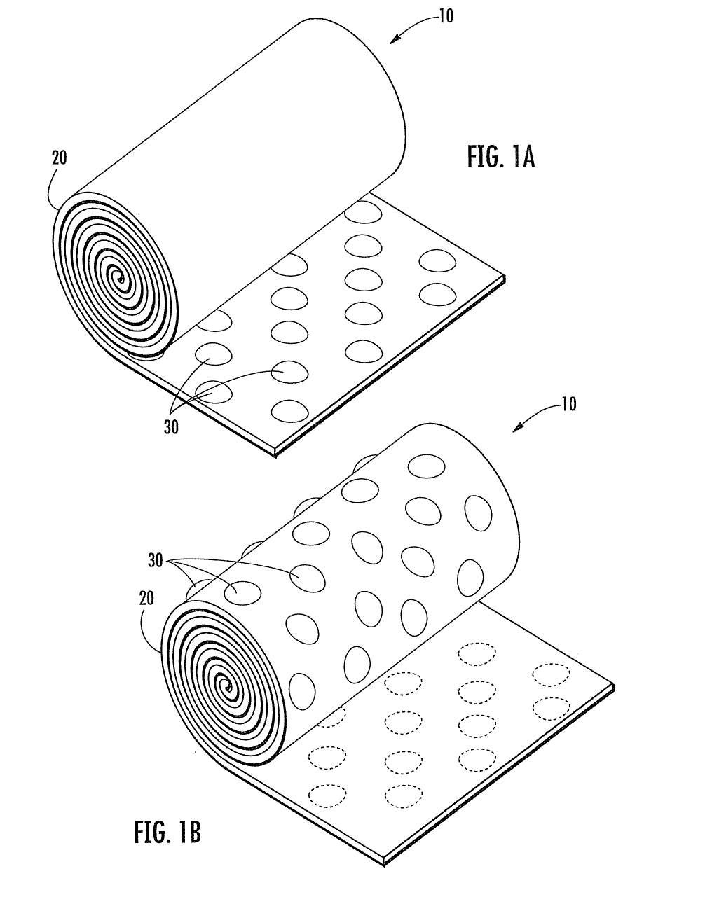

In accordance with the presently disclosed subject matter, resorbable medical implants and related methods are provided. In one aspect and with reference to FIGS. 1A and 1B, a resorbable implant, generally designated 10, can comprise a resorbable matrix 20 having at least one surface that can have or define a surface texture in at least a portion of the surface, as described further herein. Matrix 20 can be configured into a variety of shapes depending on the application for implant 10. For instance, exemplary embodiments of implant 10 in which matrix 20 is rolled into a spiral shape are shown in FIGS. 1A and 1B, although it is envisioned in accordance with this invention that the shape and form of the implant can vary (e.g., folded, stacked).

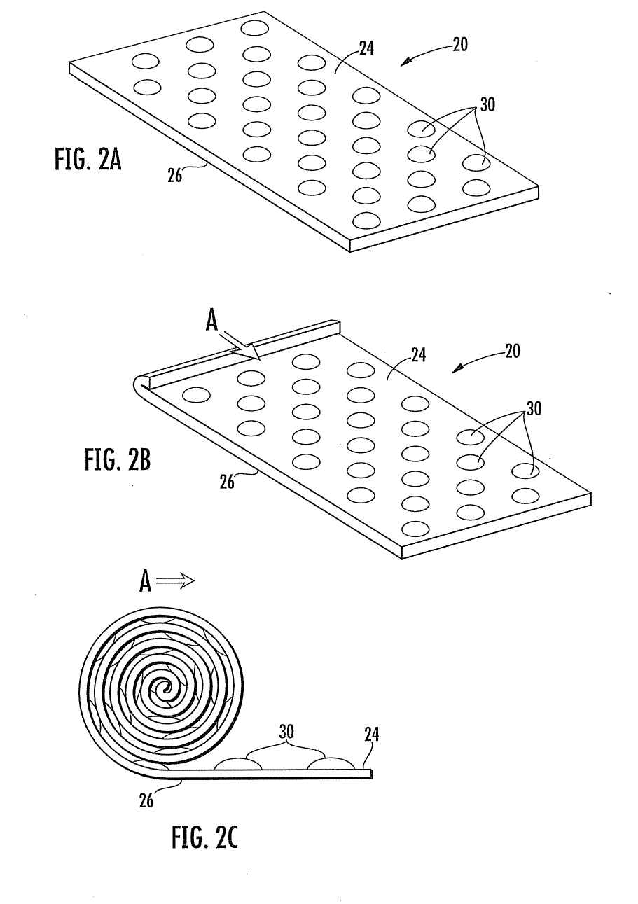

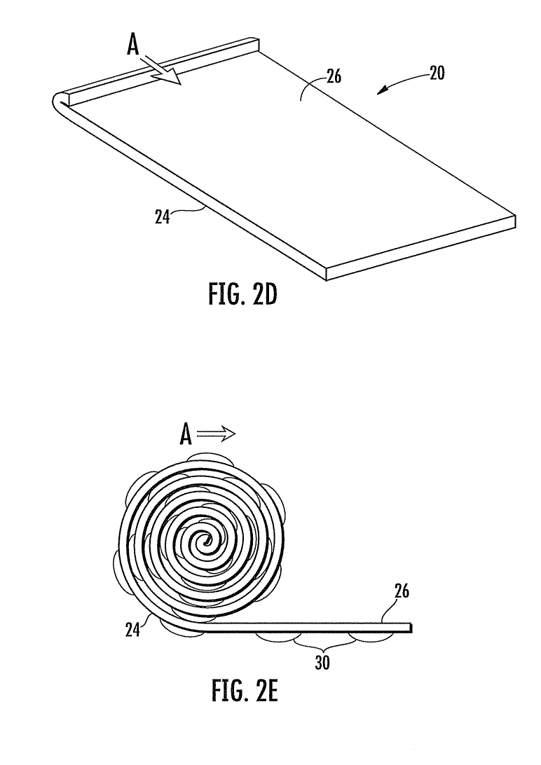

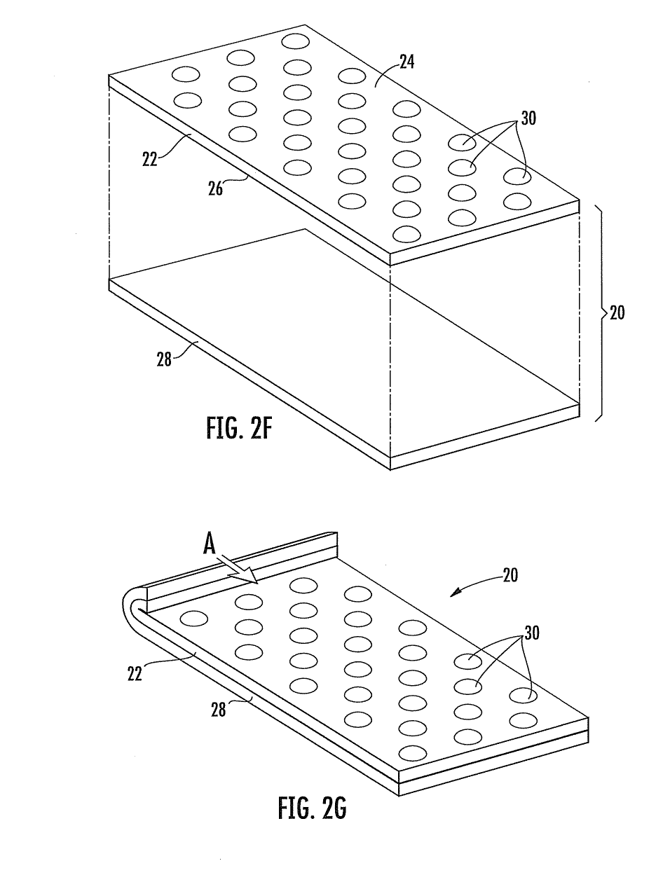

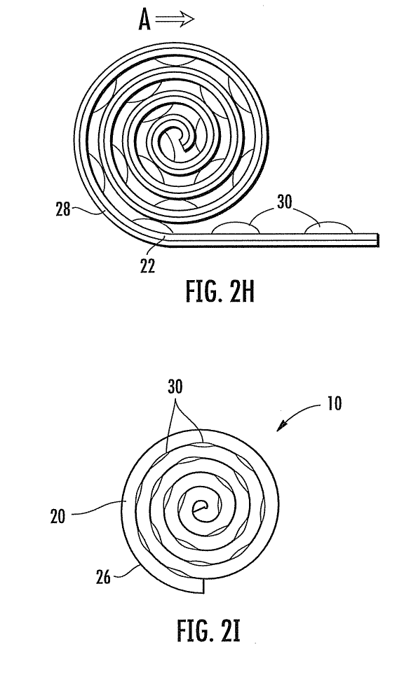

As shown in FIGS. 1A, 1B, and 2A-2I, texture can be defined or otherwise disposed on a surface of matrix 20 and can include a plurality of projections such as projections 30, grooves, ridges, grids, or any other surface modifications that increase the friction between the portions of the matrix when in contact. One or both of the top and bottom surfaces of matrix 20 can be textured. Referring in particular to FIGS. 2F-2I, matrix 20 can include one or more layers of resorbable materials. For example, the resorbable medical implant can comprise a matrix 20 formed from a first layer 22 including a first assembly of resorbable materials (e.g., collagen fibers) and a plurality of projections 30 on a top surface 24 of first layer 22. As is shown in FIG. 2F, matrix 20 can further include a second layer 28 bonded to a bottom surface 26 of first layer 22 and can comprise a second assembly of resorbable materials. The density of second layer 28 can be different from the density of first layer 22. For example, second layer 28 can have a lower density than first layer 22. Matrix 20 can then be spirally wound about itself to form implant 10.

In addition, as is shown in the embodiment depicted in FIG. 1A, matrix 20 can be rolled such that the surface defining the surface texture (e.g., surface having projections 30) can face inward in implant 10 (i.e., towards the center of implant 10) and thus contact the outer surface of the preceding turn. Alternatively, as is shown in the embodiment depicted in FIG. 1B, the surface defining the surface texture can face outward in implant 10 (i.e., away from the center of implant 10) and thus contact the inner surface of the succeeding turn. In either configuration, implant 10 can be stabilized in its rolled-up form without the need for any connector or other stabilizing means. Additional stabilizing means (e.g., adhesives) can be included if desired, however, to further support the medical implant in a spiral form.

The implant can be of a size appropriate for its intended use, for example, of a size suitable for use as an interpositional spacer in the thumb basal joint. The size of the implant can be controlled by specifically selecting the size of matrix 20 to be rolled or by removing excess matrix material once the desired size of implant 10 is achieved.

As is illustrated in FIGS. 2A-2I, a variety of exemplary processes can be used for preparing implants according to the present subject matter. First, a sheet of raw material can be provided from which matrix 20 can be formed. For instance, as discussed below, the raw material can include a Suturable DuraGen™ dural regeneration matrix (e.g., a 14″×17″ sheet), produced by Integra LifeSciences Corporation (Plainsboro, N.J.), or another resorbable material. The sheet of raw material can be cut to a size based on the desired final size of the implant. In some embodiments, this step of cutting can involve cuffing the sheet of raw material into strips having a fixed width (e.g., about 4″) and a predetermined length based on the desired size of the implant. In one particular example, two sheets of Suturable DuraGen™ matrix can be cut into strips (e.g., approximately 4″×7″ strips) such that the combined mass of the strips can be between about 6 and 7 g. In another particular example, a larger implant can be produced by cutting two sheets of Suturable DuraGen™ matrix into strips (e.g., approximately 4″×10″ strips) which can have a combined mass of between about 9 and 9.5 g. FIG. 2A illustrates one example of a strip of material serving as a matrix 20 having a top surface 24, a bottom surface 26, and plurality of projections 30 formed on top surface 24.

The raw material can further be washed to remove excess enzyme and non-collagenous impurities (e.g., protein impurities) if necessary. The material can then be compressed. For instance, multiple sheets of the material can be stacked and compressed using fixed force compression method (e.g., a Carver press applying a predetermined load of between about 1,000 and 10,000 pounds). Alternatively, the material can be compressed using a fixed gap compression method (e.g., dual roller compression). After compression, the raw material can have a density of greater than about 0.01 g/cm³, for example greater than about 0.2 g/cm³, in the range of about 0.05 g/cm³ to about 1 g/cm³, about 0.2 g/cm³ to about 0.6 g/cm³, about 0.2 g/cm³ to about 0.4 g/cm³.

Once the material is prepared in this manner, implant 10 can be formed. In one arrangement, the sheets can be separated and the individual strips of raw material can be rolled individually. As shown in FIGS. 2B and 2C, a single sheet of material can be rolled lengthwise in the direction of arrow A by tucking a free end inward toward top surface 24 and winding the sheet to form implant 10 (e.g., a solid tube). Alternatively, as is illustrated in FIGS. 2D and 2E, matrix 20 can be rolled lengthwise in the direction of arrow A to form implant 10 by tucking a free end inward toward bottom surface 26 and winding the sheet. In either configuration, a second sheet can be wound around this first implant 10, the first implant 10 of the first sheet thereby serving as a core. The second sheet can be the same size as the first sheet, or it can have dimensions that differ from the first sheet (i.e., different length or width).

In another arrangement, multiple sheets can be rolled together. For example, as is shown in FIG. 2F, matrix 20 can be formed from the stacked combination of a first layer 22 and a second layer 28. The multiple compressed layers can be rolled lengthwise in the direction of arrow A (See FIG. 2G) by tucking a free end inward and winding the sheets inwardly until implant 10 is formed (See FIG. 2H). Just as with the previous configurations, matrix 20 can be rolled in either direction depending on whether it is desired to have projections 30 face inwardly or outwardly. In yet another arrangement, a single sheet or strip of raw material can be folded and then rolled, thereby creating a “multi-layer” implant 10 with a single piece of material. Implant 10 does not need to be continuous, and it can be formed from comparatively larger/wider and smaller/narrower strips that are rolled together. Specialized manufacturing process equipment (e.g., a “sushi roller”) can be provided to assist with the rolling. In still another arrangement, multiples layers can be stacked, and the stack can be cut into a desirable size and shape for the implant. In this arrangement, the layers need not be rolled into a spiral roll.

In any arrangement, whether matrix 20 is spirally wound or folded, it is advantageous to make sure the layers are tightly compressed against each other. For example, although it is possible that minute air bubbles can be trapped between the layers in the final, fully compressed form of implant 10, matrix 20 can be wound such that there is no air space at all between any of the layers or substantially no space between the layers. Specifically, matrix 20 can be tightly wound such that projections 30 are compressed into bottom surface 26 of the next most interior winding of implant 10, and thus top surface 24 of one winding comes into contact with bottom surface 26 of the next winding. In this way, implant 10 can be formed without any space between the layers of matrix 20, as is shown in FIG. 21.

Once implant 10 of matrix 20 material is formed, implant 10 can be cut as needed to a size suitable for use as a medical implant (e.g., 20-22 mm). The cutting can be achieved using a single or plurality of parallel blades for cutting the rolled construct with either moving or stationary blades. In addition, implant 10 can be cut in any of a variety of directions, including longitudinally (e.g., creating a half-cylindrical shape) or transversely (e.g., creating short cylindrical sections), among others.

In addition, implant 10 can be dried, after which the implant can be sterilized by conventional methods, for example electronic beam or gamma sterilization. Suitable drying methods can include low pressure drying (e.g., vacuum chamber), low humidity drying (e.g., desiccators), forced convection drying (e.g., laminar flow hood), or freeze drying (e.g., lyophilization).

Accordingly, the implant according to the presently disclosed subject matter (e.g., a CMC trapezium implant) can be a single-use device packaged in double sterile barriers.

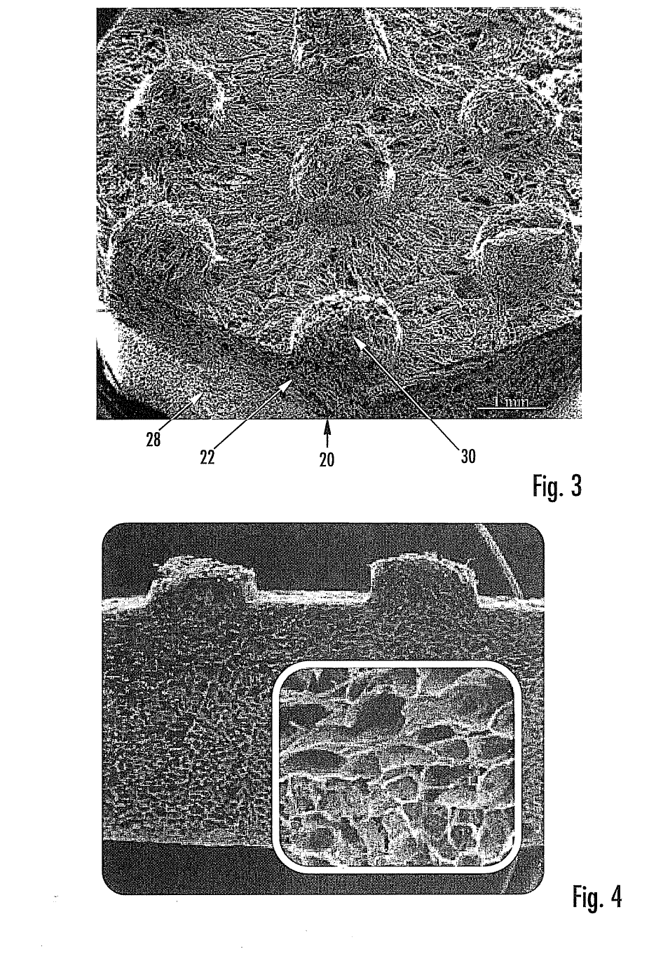

FIGS. 3 and 4 show a matrix 20 that can be used for preparing the resorbable implant. Matrix 20 can include a plurality of projections 30 and pores. As shown in FIG. 3, first layer 22 can have projections 30 and a higher density than second layer 28. The higher density of first layer 22 can provide enhanced strength and handling property to the resorbable implant. It should be noted, however, that a matrix having multiple densities is not necessary to form an implant according to the presently-disclosed subject matter.

As noted above, the presently disclosed subject matter also provides a method for using a resorbable implant for filling a void between bones, or for replacing a bone or a portion of the bone. The method can comprise providing a resorbable medical implant according to the subject matter discussed above, the resorbable medical implant being of a size appropriate for filling the void. The resorbable medical implant can be placed in the void space against a bone surface surrounding the void. More specifically, the method can include providing the implant for CMC arthroplasty for basal thumb joint disabilities. Examples of such thumb joint disabilities include thumb disabilities caused by osteoarthritis, post-traumatic arthritis, or rheumatoid arthritis.

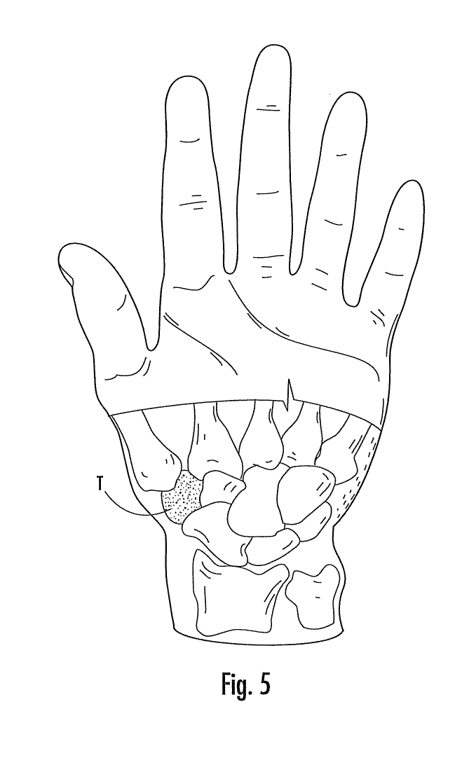

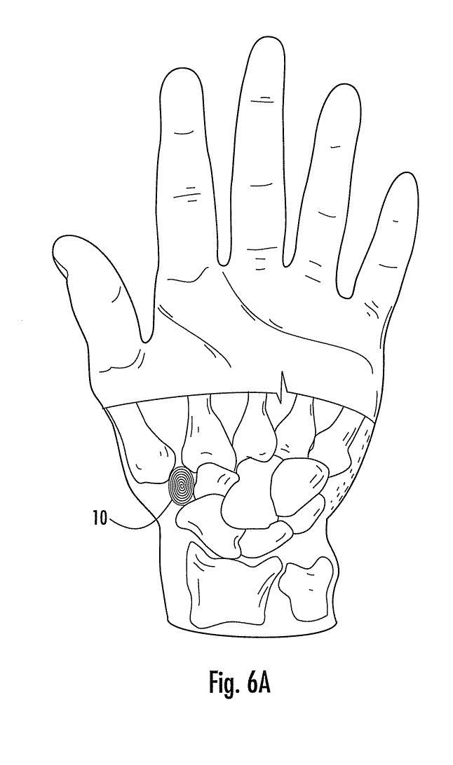

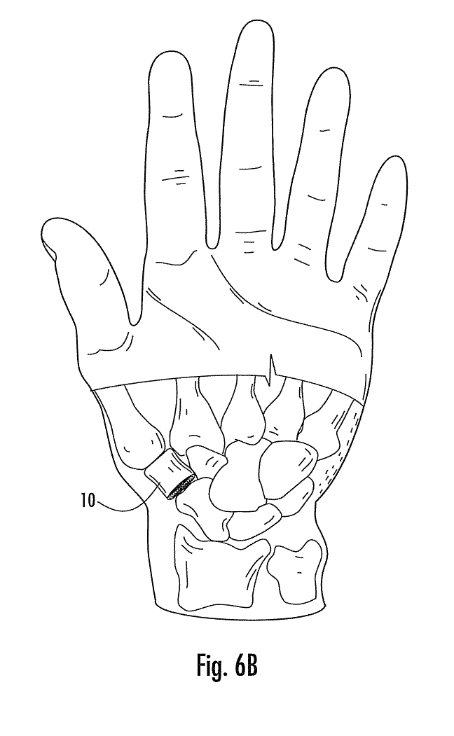

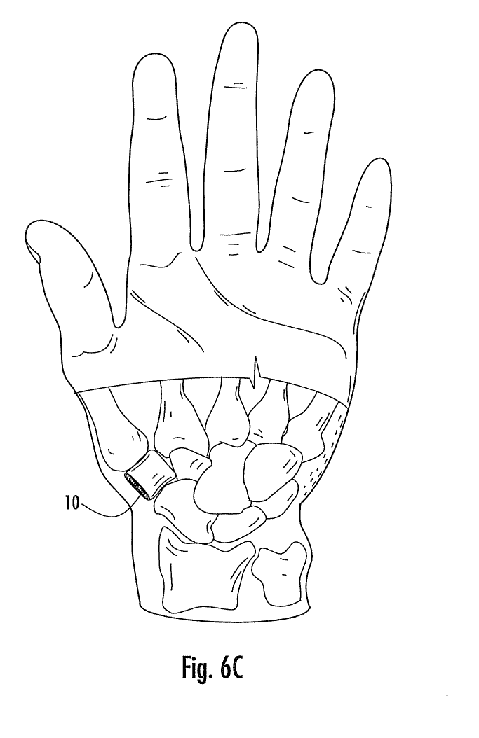

Resurfacing, partial resection, or total resection of the trapezium bone T can be performed to establish appropriate space for the implant. After partial or total resection of the trapezium bone T, as shown in FIG. 5, the implant (e.g., implant 10) can be oriented and placed in the space that becomes available as illustrated in any of FIGS. 6A through 6C. The implant can be placed in the space in the dry form, or alternatively, the implant can be wetted by a liquid (e.g., blood or saline) before use. The user can also cut the implant, unwind the implant, or unwind plus cut a portion of the strip, to produce an implant of an appropriate size to accommodate the joint space to be filled.

As noted above, implant 10 is not limited to being rolled into a spiral shape. In another embodiment, matrix 20 can be folded upon itself to form layers. Such a layered configuration for implant 10 can be used in a variety of medical applications, such as, for example, for partial trapeziectomy or implant arthroplasty procedures. In this folded configuration, matrix 20 can be hydrated with water, compressed in a manner similar to the process discussed above with respect to the rolled configuration, and then folded in half. Matrix 20 can then be further compressed and folded one or more times until a desirable thickness for implant 10 is achieved. Similarly to the spirally-wound configurations, it can be advantageous for matrix 20 to be folded such that little to no air space remains between the layers. Further, depending on the desired final shape for implant 10, the folded matrix 20 can be punched out into any of a variety of shapes (e.g., squares, circles).

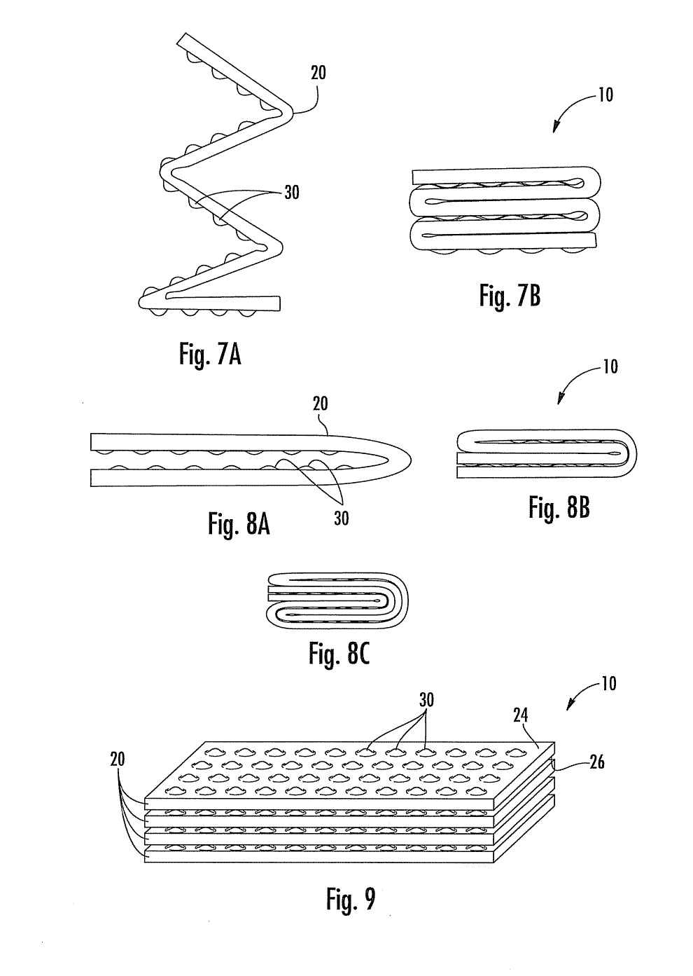

Specifically, FIGS. 7A and 7B illustrate an exemplary method for forming a folded form of implant 10. Matrix 20 can be formed from a strip of a resorbable material (e.g., a 3 cm×17 cm strip of Suturable DuraGen™ matrix), which can be hydrated and compressed (e.g., 5,000 lbs of compression). A part of the length of matrix 20 (e.g., about a 34 mm length of the 17 cm strip) can be folded over and compressed again. The folded part of matrix 20 (e.g., another 34 mm length) can be folded in an opposite direction over the first fold (i.e., “accordion style”) and compressed again. FIG. 7B shows matrix 20 having been folded 4 times (i.e., 5 layers stacked), but it is to be understood that any number of folds can be performed to achieve the desired thickness for implant 10. The folded strip can further be freeze-dried and cut into the desired size and shape (e.g., approximately 13 mm×17 mm pieces with a thickness of 3-4 mm).

Alternatively, instead of sequentially folding matrix 20 in opposing directions (i.e., “accordion style”), FIGS. 8A through 8C illustrate another exemplary method for forming a folded form of implant 10. In this alternative, approximately half of the length of matrix 20 is folded over (See FIG. 8A). At least the first fold, matrix 20 can be folded such that bottom surface 26 (i.e., the “smooth side”) of matrix 20 is on the outside, and thus projections 30 on top surface 24 face inward. Matrix 20 can further be folded in half one or more times in the same direction (See FIG. 8B). Once a number of folds are performed to achieve a desirable number of layers, matrix 20 can then be compressed to form implant 10 (See FIG. 8C). Again, the folded strip can further be freeze-dried and cut into the desired size and shape.

In yet another embodiment illustrated in FIG. 9, matrix 20 can be formed from multiple strips of a resorbable material (e.g., 4-5 strips/sheets), which can be hydrated. The multiple strips can be stacked on top of one other and compressed (e.g., 4,000-7,000 lbs of compression) to achieve a desirable thickness. Again, depending on the desired final shape for implant 10, the stacked layers of matrix 20 can be cut out into a desired shape and size (e.g., 13 mm×17 mm pieces).

Regardless of the specific form of implant 10, the material used for matrix 20 can be any suitable resorbable polymer material. The material can also be selected to be physiologically compatible (i.e., non-inflammatory, non-adhesion inducing, etc.), sufficiently noninfectious (i.e., decontaminated, etc.) to prevent the transmission of viruses, pliable, available in a variety of sizes, high in tensile strength, and/or inert. In addition, the material can be sufficiently pliable when wet so as to conform to a surface of an underlying tissue. For example, some varieties of human and animal tissues as well as certain synthetic polymer materials can provide many of these advantages. In particular, materials that can commonly be used as dural substitutes for repairing damaged meningeal membranes of the brain and spinal cord can also provide these kinds of advantages. One example of such a dural substitute is a product marketed by Integra LifeSciences Corporation (Plainsboro, N.J.), under the trademark DuraGen. The dural substitute product, which is described in U.S. Pat. No. 5,997,895, comprises a collagen matrix.

A further example of a material that can be used for matrix 20 is disclosed in U.S. patent application Ser. No. 11/622,695, the disclosure of which is incorporated by reference in its entirety. As is disclosed in that application, matrix 20 can comprise at least two layers, each of the layers comprising resorbable materials (e.g., collagen fibers). In particular, a first layer 22 can have a relatively high density and relatively small pores, whereas a second layer 28 can have a relatively low density and relatively large pores. The walls of the pores in first layer 22 can be thicker than the walls of the pores in second layer 28. The at least two layers can be bonded together to form an integral matrix 20.

First layer 22 can comprise a relatively dense assembly of resorbable materials. For instance, first layer 22 can be formed as a non-woven assembly of fibers. Alternatively, first layer 22 can be formed using other fiber assemblies, including woven fiber assemblies. Matrix 20 can also be provided in the form of a combination of different fiber assemblies. In such an embodiment, all of the fiber assemblies need not be sufficiently porous to promote tissue growth therethrough. In certain exemplary embodiments, other optional additives can also be present in the matrix in addition to collagen, for example, growth factors such as bone morphogenic protein (BMP), antimicrobials, materials that promote fibrous tissue growth, and the like.

The density of first layer 22 can be designed to decrease along a gradient from top surface 24 to bottom surface 26. (See, e.g., FIG. 4) First layer 22 can have a density of about 0.005 mg/mm³ to 0.4 mg/mm³. For instance, the density can be between about 0.01 mg/mm³ to 0.1 mg/mm³, in some instances between about 0.025 mg/mm³ to 0.075 mg/mm³.

The size of the pores of first layer 22 preferably can increase along a gradient from top surface 24 to bottom surface 26. Pores of top surface 24 of first layer 22 comprise pore sizes of about 10 μm to 250 μm. In particular embodiments, the pore sizes can range from about 30 μm to 150 μm, or in some cases between about 50 μm to 80 μm. Likewise, pores of bottom surface 26 of first layer 22 can comprise pore sizes of about 10 μm to 500 μm, particularly between about 50 μm to 150 μm, more particularly from about 80 μm to 120 μm. It should be noted that these parameters refer generally to properties of the raw material, and thus it should be understood that the densities and surface characteristics of implant 10 can be altered by subsequent processing steps, discussed below.

The relatively high density and relatively low porosity of first layer 22 can provide matrix 20 with improved strength relative to certain prior art products. The strength of first layer 22 (and/or the entirety of matrix 20) can be quantified by measuring the tear strength using any improvised or standard protocol for measuring tear strength in non-woven materials, such as ISO 9073-4: 1997, ASTM 5733, ASTM 5734 or ASTM 5735. In some embodiments, the wet tear strength (i.e., the tear strength of a specimen measured within 5 minutes following a 5-minute immersion in a normal saline solution) can be at least 1.5 N, more preferably at least 2.5 N, as measured by an Instron tensile testing machine. Enhanced tear strength renders the matrixes of the present subject matter suitable for suturing to adjacent tissues, although sutures are not required to attach matrix 20 to such tissues.

Top surface 24 of first layer 22 preferably includes at least one projection 30, and more preferably includes a plurality of projections 30. The size and shape of projections 30 are not particularly limited. Rather, the quantity, size, shape, and placement of projections 30 can be dictated by the first layer preform compression step of the matrix forming method. In particular, in the first layer compression step, fluid can be drained from the first layer preform by compressing it against a mold having one or more drainage outlets. Portions of top surface 24 that enter the outlets can be molded by compression to form projections 30. Thus, the quantity, size, shape and placement of projections 30 can be adjusted by selecting a mold having the desired number of drainage outlets (i.e., holes), which have the desired dimensions. The configuration and distribution of the drainage outlets can be modified to produce a matrix having a desired surface texture.

For instance, the number of projections 30 that can be formed on the surface of matrix 20 can range from about 1 to 500 projections per square centimeter. In many embodiments, however, the number of projections 30 can be between about 5 and 100 per square centimeter, more particularly between about 10 and 20 projections per square centimeter. In addition, projections 30 can be sized to have an average diameter ranging from about 0.001 to 0.5 cm, with many embodiments having average diameters between about 0.05 and 0.3 cm, and in some cases projections 30 can have diameters ranging between about 0.1 and 0.15 cm. Average heights of projections 30 can likewise vary between about 0.001 and 0.2 cm, with many embodiments having average heights between about 0.02 and 0.2 cm, and particular embodiments having average heights of about 0.04 to 0.08 cm.

In addition to being raised above top surface 24 of first layer 22, projections 30 can be less dense and more porous than the adjacent areas. The outlets in the mold through which projections 30 are permitted to expand to relieve the compressive force experienced by projections 30, resulting in projections 30 that can be less dense than adjacent areas of first layer 22. Projections 30 can therefore be more permeable to fluid and tissue infiltration than adjacent portions of first layer 22.

Second layer 28 can be bonded to the bottom portion of first layer 22. The term “bonded” as used herein is synonymous with the term “attached”. Thus, the nature of the bonding is not intended to be limited to a particular method of attachment. For instance, the two layers can be bonded together by intermingled resorbable materials. In this regard, the use of adhesives is not required, but adhesives can be used to assist in bonding the layers together.

Second layer 28 can comprise a second, preferably non-woven, assembly of resorbable material fibers having a second density of about 0.001 mg/mm³ to about 0.12 mg/mm³, with many embodiments exhibiting densities of about 0.005 mg/mm³ to 0.1 mg/mm³, and particular embodiments having densities ranging from about 0.009 mg/mm³ to 0.05 mg/mm³. Regardless of the density of the fibers, the pore sizes in second layer 28 can range from about 10 μm to about 500 μm, with many embodiments having pore sizes in the range of about 50 μm to about 150 μm, with surface pores being smaller than cross-sectional (internal) pores. Further, the pore sizes can very throughout the thickness of second layer 28. For instance, the pore size of surface pores can range from about 30 μm to about 150 μm (e.g., about 70 μm), and the pore size of cross-sectional pores ranges from about 50 μm to about 300 μm (e.g., about 150 μm). Again, it should be understood that the parameters listed above refer generally to properties of the raw material, and thus the densities and surface characteristics of implant 10 can be altered by subsequent processing steps, discussed below.

Although a two-layered matrix 20 is described in detail herein, it is also within the scope of the current subject matter to provide a matrix 20 having three, four, five, or more layers. These additional layers can comprise resorbable material fibers assembled as a non-woven, woven, or film. Matrix 20 can be provided in the form of a composite of any two or more of the foregoing forms.

For instance, a third layer can be added to the first and second layers 22 and 28 described above, wherein the third layer can be substantially similar to first layer 22, and second layer 28 can be sandwiched between the first and third layers. The third layer can be a non-woven assembly of resorbable material fibers bonded to the bottom surface of the second assembly.

Regardless of the specific configuration of layers used to form the matrix, matrix 20 can be a planar object having pores of a sufficient size and quantity to permit growing tissue to infiltrate the matrix. The length and width of matrix 20 can be dictated by its intended use. Certain embodiments of matrix 20 can be about 1-15 cm in length and width. The thickness of matrix 20 can be related to the number of layers, the height of any projections 30 and the density of each layer. For example, certain embodiments of matrix 20 can have a thickness of about 0.1 mm-20 mm, for example, about 0.2 mm-10 mm, about 1 mm-6 mm, about 2 mm-4 mm.

Matrix 20 can optionally be cross-linked with heat or a suitable chemical cross-linking agent. (See, e.g., Chemistry of Protein Conjugation and Crosslinking, Wong, ed., CRC Press, 1993). For example, matrix 20 can be cross-linked by exposure to vapors from an aqueous formaldehyde solution (e.g., a solution having a 9.6% formaldehyde concentration) for about ninety minutes at about 25° C., followed by forced air ventilation for about one hour.

The method for producing matrix 20 of the present subject matter makes use of steps that are recognized as being effective for inactivating viral and prion contamination. This gives matrix 20 a very high safety level while eliminating the inflammatory response. That is, the method for producing matrix 20 of the present subject matter provides a matrix 20 that is substantially free of viruses and prions without being physiologically incompatible. Stated otherwise, matrix 20 can be provided such that it does not contain infection-effective amounts of viruses and prions. More specifically, matrix 20 can comprise a resorbable material (e.g., collagen) treated by a process sufficient to achieve at least a 4 log clearance of virus, more preferably at least a 6 log clearance of virus, and even more preferably at least an 8 log clearance of virus, as measured with a statistical confidence level of at least 95%. For example, if the concentration of virus before treatment is 10⁷ and after treatment is 10¹, then there has been a 6 log clearance of virus.

In preparing matrix 20 of the presently disclosed subject matter, a first mixture of resorbable materials and a first liquid carrier can be provided. The mixture can be a collagen dispersion prepared in a manner well known in the art. One such preparation is taught in U.S. Pat. No. 3,157,524. Another suitable preparation of collagen is taught in U.S. Pat. No. 3,520,402. A further example of a suitable preparation of collagen can include a native source of Type I collagen (e.g., skin, tendons, ligaments or bone), which can be first mechanically or hand cleaned of fat, fascia, and/or other extraneous matter and washed. The cleaned and washed collagen containing material can then be comminuted, generally by slicing or grinding.

The material can then be subjected to an enzyme treatment while under intermittent stirring with a proteolytic enzyme (e.g., ficin, pepsin, or the like) so as to remove non-collagenous impurities that may cause antigenic activity and to swell the collagen by removing elastin. The amount of enzyme added to the collagen material and the conditions under which enzyme digestion takes place can depend upon the particular enzyme being used. For example, when using ficin, the pH can be adjusted to about 6.0 to 6.3, and the collagen material can be digested for about 1 to 2 hours at a temperature of about 36.5° C. to 37.5° C. with one part ficin for every 150 parts of collagen material. After the requisite amount of time, the enzyme can be inactivated by appropriate means well known in the art, such as by the addition of a solution of an oxidizing agent (e.g., sodium chlorite). The enzyme treated collagen containing material can then be washed to remove excess enzyme and the non-collagenous protein impurities. For instance, the washing can be carried out with ultrafiltered and deionized water and optionally further washed with dilute aqueous hydrogen peroxide.

In one particular embodiment of the present subject matter, the enzyme digested collagen containing material can be further subjected to an alkali treatment at a pH of about 13 to 14, at a temperature of about 25° C. to 30° C. for a period of about 40 hours (e.g., between about 35 to 48 hours). Suitably, the alkali treatment can be carried out in an aqueous solution of 5% sodium hydroxide and 20% sodium sulfate. This alkali treatment removes contaminating glycoproteins and lipids. The solution can then be neutralized with a suitable acid (e.g., aqueous sulfuric acid) and thoroughly washed.

The collagen material can then be further swollen with a suitable acid solution that does not cause any cross-linking of the collagen. Such acids are well known to those skilled in the art and include acetic acid, hydrochloric acid, lactic acid, and the like. Regardless of which acid is used, the pH of the acid collagen dispersion can be in the range of about 2 to 3.

The dispersed collagen mixture can then be homogenized by any conventional means, such as a blender or homogenizer, so as to further dissociate the fibers. The homogenized mixture can be filtered to remove unswollen, non-collagenous material by means well known in the art, such as bypassing the dispersion through a 100 mesh stainless steel screen. The resulting filtered collagen dispersion can then be used to prepare matrix 20 of the present subject matter.

Alternatively, physiologically compatible collagen that is substantially free of active viruses and prions can be obtained from transgenic animals bred for the purpose of synthesizing human collagen in a readily harvestable form. (See, e.g., U.S. Pat. No. 5,667,839 to Berg.) Since transgenic animals can be bred and maintained in controlled environments, which prevent them from carrying infections which must be inactivated, the collagen harvested therefrom can be physiologically compatible and substantially free of active viruses and prions without further treatment. That being said, further treatment can be performed for an added measure of safety.

In one exemplary embodiment, the collagen can be lactic-acid-derived collagen fibers. Such fibers can be produced by a process comprising dispersing a virus- and prion-free collagen source (e.g., alkali-treated bovine tendon slices) in an aqueous solution of lactic acid (e.g., about 85% lactic acid), homogenizing the dispersion, filtering the homogenized lactic acid dispersion, and precipitating collagen fibers from the homogenized lactic acid dispersion by addition of aqueous ammonium hydroxide (e.g., about 0.35% ammonium hydroxide) sufficient to adjust the pH to about 4.6-4.9.

Lactic-acid-derived/ammonium-hydroxide-precipitated collagen fibers can be much longer than fibers produced by mechanical or chemical disruption of raw bovine tendon material, and these longer fibers provide greater strength to the final product. This difference is thought to occur because during ammonium hydroxide precipitation, the collagen fibers re-coil and are therefore longer. The enhanced strength of products of the present subject matter produced according to this particular method can be sufficiently strong to be watertight and suturable without the need for cross-linking, thus allowing the degree of cross-linking to be selected based on the desired rate of bioresorption.

The collagen fibers can further be dispersed in water to provide the first mixture. In certain embodiments, the first mixture can be a dispersion in accordance with the teachings of U.S. Pat. No. 4,963,146. The first mixture can be cast on a first mold, such as a perforated tray made of a metal (e.g., aluminum). The mold can comprise a non-stick coating. The quantity and dimensions of perforations through the mold can be selected to provide matrix 20 with desired characteristics and/or to achieve a desired drainage rate of liquids from the mold.

An initial amount of the liquid carrier in the first mixture on the first mold can then be drained by gravity through the perforation(s) to provide a first layer 22 preform on the mold. First layer 22 preform can be frozen and then thawed to release additional moisture. The thawed preform can then be compressed against the first mold to drain through the first mold an additional amount of the first liquid carrier and provide first layer 22.

Compression can be accomplished by sandwiching the thawed preform between two substantially identical molds. The first and/or second mold can be pressed against the thawed preform in a direction substantially perpendicular to the planes defined by the surfaces of the molds in contact with the preform. Where the second mold has drainage outlets, the first liquid carrier can drain through both the first and second molds, and the resulting first layer 22 can have projections 30 on top surface 24 and/or bottom surface 26. The bottom projections 30 can ultimately be obscured by second layer 28 bonded to bottom surface 26 of first layer 22.

In addition, the amount of pressure applied in compression can be selected such that the resulting first layer 22 has the desired density and porosity. The collagen matrix 20 can be compressed from approximately 10 mm thickness to about 1.5 mm (e.g., between about 1-2 mm). First layer 22 can be provided with the pore size ranges discussed above.

In some embodiments, a second mixture of collagen fibers and a second liquid carrier can be cast on first layer 22 in the first mold. The collagen content in the aqueous dispersion can be controlled at between about 0.5-1.0% w/v with a nominal target of about 0.75% w/v. The second mixture can be identical to or different from the first mixture. In certain embodiments, the first and second mixtures can be drawn from a common source (e.g., a stock slurry). In certain embodiments, the second mixture can be a dispersion in accordance with U.S. Pat. No. 5,997,895. The second mixture can then be lyophilized with first layer 22 to provide second layer 28 on first layer 22 and thereby provide the matrix. In alternative embodiments, second layer 28 can be formed as an independent component and then bonded to first layer 22 using adhesive or other means.

Further layers in addition to first and second layers 22 and 28 can also be incorporated into matrix 20 in certain embodiments. Such additional layers can be formed on the underlying layer or formed independently of matrix 20 and bonded to the underlying layer. For example, a third layer can be provided on second layer 28 as follows. First, a third mixture of collagen fibers and a third liquid carrier can be provided. The mixture can be a dispersion of collagen fibers in water. The third mixture can be identical to or different from the first and/or second mixtures. In certain embodiments, the first, second and/or third mixtures can be drawn from a common source.

The third mixture can be cast on a second mold, the second mold being either identical to or different from the first mold. An initial amount of the third liquid carrier can be permitted to gravity drain through the second mold to create a third layer preform. The third layer preform can then be frozen and thawed to drain additional liquid therefrom. The thawed third layer preform can then be compressed against the second mold to drain through the second mold an additional amount of the third liquid carrier and provide the third layer. This step can be performed in a manner identical to that used to form first layer 22.

The third layer can be compressed such that it has a third layer target porosity equivalent to, of a greater porosity than, or of a lesser porosity than one of the other two layers. For example, to provide a three-layered system with the outer two layers comprising a dense tensile matrix, the third layer can contain pores having a pore size within the ranges discussed above with respect to first and second layers 22 and 28. The third layer can then be placed on the second mixture cast on first layer 22 prior to lyophilizing the second mixture, to bond second layer 28 to the first and third layers, and thereby provide a three-layered matrix.

In a second example of a three-layered matrix, the dense tensile layer can be contained within two outer layers of low density matrix. In this embodiment, the third layer can be cast as a dispersion either independently or directly upon the preceding two layers and can be bonded by a lyophilization process to the inner matrix. In this example, the third layer can comprise a second non-woven assembly of collagen fibers having a density of about 0.0001 mg/mm³ to about 0.12 mg/mm³ and a pore size of about 50 μm to about 150 μm.

The collagen used in the matrixes of the present subject matter can be at least about 80% pure, substantially free of all prion and viral contamination, has less than 0.03 eu/gm endotoxins, has not more than 5% fat content, has at least 10% hydroxyproline content, and has not more than 5% ash content. It is thought that suitable collagen can be derived from bovine corium or bovine tendon collagen, but the collagen can be obtained from other sources, including other bovine tissues and tissues from other animals, including non-bovine mammals, non-mammalian animals and transgenic animals.

In addition to collagen, certain embodiments of matrix 20 can include natural and/or synthetic polymers for structural support. The polymers can be biocompatible and/or bioresorbable. Suitable polymers include but are not limited to biocompatible and/or bioresorbable lactides, glycolides, and copolymers thereof, polycaprolactones, polyethylene carbonate, tyrosine polycarbonates, tyrosine polyacids, and polyanhydrides. The molecular weight of the polymer can be selected to range from about 5,000 to about 500,000.

Matrix 20 can be nonantigenic in addition to being noninfectious and physiologically compatible. In addition, matrix 20 can be substantially resorbed.

The present subject matter can be embodied in other forms without departure from the spirit and essential characteristics thereof. The embodiments described therefore are to be considered in all respects as illustrative and not restrictive. Although the present subject matter has been described in terms of certain preferred embodiments, other embodiments that are apparent to those of ordinary skill in the art are also within the scope of the present subject matter.

What is claimed is:

1. A resorbable medical implant, comprising:
a matrix including a first layer containing a first assembly of resorbable materials, the first layer defining a top surface and a bottom surface; and
a surface texture on the top surface of the first layer of the matrix;
wherein the top surface of a first portion of the matrix is compressed against at least a second portion of the matrix to form a compressed matrix.

2. The resorbable medical implant of claim 1, wherein the first assembly of resorbable materials comprises a first assembly of collagen fibers.

3. The resorbable medical implant of claim 1, wherein the surface texture comprises a plurality of projections.

4. The resorbable medical implant of claim 3, wherein the plurality of projections comprises between about 10 and 20 projections per square centimeter.

5. The resorbable medical implant of claim 1, wherein the matrix is spirally wound about itself such that the compressed matrix is a spiral roll.

6. The resorbable medical implant of claim 5, wherein the surface texture is oriented inwardly towards the center of the spiral roll.

7. The resorbable medical implant of claim 5, wherein the surface texture is oriented outwardly away from the center of the spiral roll.

8. The resorbable medical implant of claim 5, wherein the matrix is spirally wound about itself by tucking an end of the matrix inwardly and winding the matrix to form a spiral roll.

9. The resorbable medical implant of claim 1, wherein the matrix is folded to form the compressed matrix.

10. The resorbable medical implant of claim 1, wherein density of the first layer decreases from the top surface to the bottom surface.

11. The resorbable medical implant of claim 1, further comprising a second layer containing a second assembly of resorbable materials, the second layer being bonded to the bottom surface of the first layer.

12. The resorbable medical implant of claim 11, wherein the second assembly of resorbable materials comprises a second assembly of collagen fibers.

13. The resorbable medical implant of claim 11, wherein the density of the second layer is less than the density of the first layer.

14. A resorbable medical implant, comprising:
a matrix including a first layer containing a first assembly of resorbable materials, the first layer defining a top surface and a bottom surface, and a second layer containing a second assembly of resorbable materials, the second layer being bonded to the bottom surface of the first layer, the density of the second layer being different than the density of the first layer; and
a plurality of projections on the top surface of the first layer of the matrix;
wherein the top surface of a first portion of the matrix is compressed against at least a second portion of the matrix to form a compressed matrix.

15. A method for making a resorbable medical implant, comprising:
providing a matrix including a first layer containing a first assembly of collagen fibers, the first layer defining a top surface and a bottom surface;
forming a surface texture on the top surface of the first layer of the matrix; and
compressing the top surface of a first portion of the matrix against at least a second portion of the matrix to form a compressed matrix.

16. The method of claim 15, wherein providing a matrix comprises bonding a second layer to the bottom surface of the first layer, the second layer comprising a second assembly of resorbable materials, the second layer having a lower density than the first layer.

17. The method of claim 15, wherein forming the surface texture comprises forming a plurality of projections.

18. The method of claim 17, comprising forming the plurality of projections of between about 10 and 20 projections per square centimeter.

19. The method of claim 17, wherein forming a plurality of projections comprises:
compressing the first layer against a mold having one or more drainage outlets, wherein portions of the top surface of the first layer enter the drainage outlets; and
molding the portions of the top surface of the first layer that enter the drainage outlets to form the projections.

20. The method of claim 19, wherein compressing the first layer comprises using one of a fixed gap compression method or a fixed force compression method.

21. The method of claim 15, wherein compressing to form a compressed matrix comprises winding the matrix about itself to form a spiral roll.

22. The method of claim 21, wherein winding the matrix comprises tucking an end of the matrix inwardly and winding the matrix to form a spiral roll.

23. The method of claim 22, wherein tucking an end of the matrix inwardly comprises tucking an end of the matrix towards the top surface, wherein the surface texture is oriented inwardly towards the center of the spiral roll.

24. The method of claim 22, wherein tucking an end of the matrix inwardly comprises tucking an end of the matrix towards the bottom surface, wherein the surface texture is oriented outwardly away from the center of the spiral roll.

25. The method of claim 15, wherein compressing to form a compressed matrix comprises folding the matrix.

26. The method of claim 15, further comprising soaking the matrix with a liquid to create a wet matrix and compressing the wet matrix to create a compressed matrix prior to winding the matrix.

27. The method of claim 26, further comprising drying the compressed matrix.

28. The method of claim 27, wherein drying the compressed matrix comprises low pressure drying, low humidity drying, forced convection drying, and/or freeze drying.

29. The method of claim 15, further comprising cutting the compressed matrix to a predetermined size and shape.

30. A method for filling a void between bones, comprising:
providing a matrix including a first layer containing a first assembly of resorbable materials, the first layer defining a top surface and a bottom surface, a surface texture being on the top surface of the first layer of the matrix, the top surface of a first portion of the matrix being compressed against at least a second portion of the matrix to form a compressed matrix; and
positioning the compressed matrix against a bone surface in a void space between bones.

31. The method of claim 30, wherein positioning the compressed matrix comprises positioning the compressed matrix in a void space between bones in the carpometacarpal joint.

32. The method of claim 31, comprising excising the trapezium bone prior to positioning the compressed matrix.

33. The method of claim 30, wherein the surface texture comprises a plurality of projections.
